(19) United States
(12) Patent Application Publication
(10) Pub. No.: US 2024/0125780 A1
(43) Pub. Date: Apr. 18, 2024

(54) METHOD FOR DETECTING ANTI-CAR ANTIBODY AGAINST HUMANIZED CART CELLS

(71) Applicant: UNION HOSPITAL TONGJI MEDICAL COLLEGE HUST, Wuhan (CN)

(72) Inventors: Heng MEI, Wuhan (CN); Yu HU, Wuhan (CN); Zhongpei HUANG, Wuhan (CN); Chenggong LI, Wuhan (CN); Wenjing LUO, Wuhan (CN)

(73) Assignee: UNION HOSPITAL TONGJI MEDICAL COLLEGE HUST, Wuhan (CN)

(21) Appl. No.: 18/459,076

(22) Filed: Aug. 31, 2023

(30) Foreign Application Priority Data
Oct. 18, 2022 (CN) 202211270320.X

(51) Int. Cl. G01N 33/569; G01N 33/58

(52) U.S. Cl. CPC G01N 33/56972; G01N 33/582; G01N 2469/20

(57) ABSTRACT

Some embodiments of the disclosure provide a method for detecting an anti-chimeric antigen receptor (CAR) antibody against humanized CAR T cells. In some examples, the method include following steps. S1. Transfecting a humanized CAR into Chinese hamster ovary (CHO) cells into using lentivirus to generate CHO cells expressing CAR as target cells. S2. Collecting samples to be tested. S3. Preparing different concentrations of standards using human proteins corresponding to a target of the CAR and establishing a standard curve. S4. Incubating the target cells with the different concentrations of standards and the samples to be tested, and capturing the anti-CAR antibody. S5. Incubating a phycoerythrin (PE)-conjugated anti-human IgG Fc antibody with CHO cells for labeling. S6. Calculating the anti-CAR antibody by detecting average fluorescence intensities of the standards, blank control, and the samples to be tested using flow cytometry.

### CROSS REFERENCE TO RELATED APPLICATIONS

This application claims priority to Chinese application number 202211270320.X, filed on Oct. 18, 2022, the disclosure of which is incorporated by reference herein in its entirety.

### FIELD OF THE DISCLOSURE

The disclosure relates generally to the field of medical technology. More specifically, the disclosure relates to methods for detecting anti-CAR antibodies against humanized CAR T cells.

### BACKGROUND

At present, chimeric antigen receptor T-cell immunotherapy (CAR-T) is widely used in the clinical treatment of diseases such as hematological tumors and solid tumors. With the improvement of technology and advancing clinical trials, a variety of chimeric antigen receptor (CAR) T-cell products have been launched one after another and more types of CAR T cells adapted to different diseases are also being released to the market one after another. For all CAR-T, the efficacy, persistence and recurrence factors have been objectives that need to be overcome and explored urgently. Particularly, the production of humoral immune antibodies against CAR (anti-CAR antibodies) may have an important impact on the above three aspects, resulting in poor clinical results. In particular, with the increasing popularity of CAR-T, more clinical cases may involve the second re-infusion of CAR T cells, and there comes more importance of the impact and detection of anti-CAR antibodies.

The single-chain variable region (scFv), the most important antigen-binding domain of the chimeric antigen receptor carried by CAR T cells, was originally derived from animals, including mouse (FMC63), alpaca, etc. Due to the differences in species, CAR as an exogenous protein will inevitably underline the possibility of inducing the anti-CAR antibodies after being infused back into the human body along with CAR T cells. Of the CARs, human anti-mouse antibody (HAMA) was among the most studied. At present, there have been complete and accurate detection methods (such as ELISA) to quantify HAMA in the plasma of patients. B y comparing the HAMA levels at baseline and during treatment follow-up, we can know about the anti-CAR antibody production against mouse-derived CAR T-cell products. In order to avoid the anti-CAR immune response due to the strong immunogenicity of animal-derived scFv, humanized CAR-T has begun to be widely studied. Initially, only the CAR fragments were humanized, and now there are many fully humanized CARs. Although species differences are eliminated, they are still human-edited and assembled sequences. For many patients, they are still exogenous proteins. Therefore, the CAR itself and its hinge region can still possibly cause anti-CAR antibodies, as also confirmed by related clinical trials.

Relevant methodologies have been established for the detection of anti-CAR antibodies against humanized CAR T cells, which is used as an indispensable detection indicator in CAR-T clinical research. These methods mainly involve the combination of anti-CAR antibodies in patient plasma by flow cytometry and determination (see XU J, et al., Proc Natl Acad Sci USA, 2019, 116(19):9543-51). Here, the transfected Chinese hamster ovary (CHO) cells expressing the corresponding CAR were incubated with the plasma to capture the anti-CAR antibody in the plasma. Then the CHO cells were labeled with PE-conjugated anti-human IgG Fc antibody, and the proportion of PE-positive cells is detected, which reflects the strength of anti-CAR antibody. However, this methodology can only judge the change of anti-CAR antibody by comparing the positive rates of cells corresponding to the baseline and post-treatment plasma samples of the same patient. Because the density of CAR on CHO cells and the number of binding anti-CAR antibody are not artificially controlled and cannot be detected, this method cannot yet realize the quantification of anti-CAR antibodies and the comparison of levels between patients. Moreover, since normal samples are not detected, the detection limit for patients and ordinary people cannot be distinguished. Therefore, we propose a method for detecting anti-CAR antibodies against humanized CAR T cells.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a method for detecting an anti-CAR antibody against humanized CAR T cells. In some examples, the method includes the following steps.

Step S1, transfecting a humanized CAR into CHO cells using lentivirus to generate CHO cells expressing CAR as target cells.

Step S2, collecting samples to be tested.

Step S3, preparing different concentrations of standard products using human proteins corresponding to the target of the CAR and establishing a standard curve.

Step S4, incubating the target cells with the different concentrations of standards and the samples to be tested, and capturing the anti-CAR antibody.

Step S5, incubating a phycoerythrin (PE)-conjugated anti-human IgG Fc antibody with the CHO cells for labeling.

Step S6, calculating a concentration (nM) of the anti-CAR antibody by detecting average fluorescence intensities of standards, blank control, and the samples to be tested using flow cytometry.

In an embodiment of the disclosure, the step S2 includes collecting 1 ml of venous blood from normal people and patients using a blood collection tube containing EDTA anticoagulant, centrifuging the venous blood to obtain plasma, and freezing the plasma at −70° C. until use, avoiding repeated freezing and thawing.

In an embodiment of the disclosure, the step S4 includes adding 20 μl of the standard, the blank control, and the plasma sample to corresponding EP tubes, mixing with the CAR-CHO cells, and incubating a resulting mixture at 4° C. for 30 minutes.

In an embodiment of the disclosure, the step S5 includes adding a PBS solution to the EP tube to wash the cells once, conducting centrifugation and discarding a supernatant, adding an appropriate amount of a fresh PBS solution and 5 μl IgG Fc-PE antibody (0.5 mg/ml) to each tube, and mixing evenly for 4 minutes and incubating for 15 minutes.

In an embodiment of the disclosure, the step S6 includes calculating a protein molar concentration (nM) of each standard using molecular weight of human CD19 protein and protein concentrations of standards 1 to 5.

The average fluorescence intensity of the blank control are subtracted from the average fluorescence intensity of all standards to obtain corresponding ΔMFI, the protein molar concentrations and ΔMFI of standards 1 to 5 are converted into 1 g values, and the 1 g values are fitted into a linear standard curve to create the formula: y=0.7253x+3.8192. In this formula, x is the 1 g value of protein molar concentration, and y is the 1 g value of ΔMFI. The protein molar concentration and ΔMFI of all samples to be tested are all converted into 1 g values, substituted into the standard curve formula, and finally the molar concentration of anti-CAR antibody for each sample is calculated.

In an embodiment of the disclosure, the humanized CAR is selected from various humanized CARs that have been put into clinical trials at present, and the targets are CD19, BCMA, CD20, CD22, or CS1.

In an embodiment of the disclosure, the sample to be tested comes from a subject who has been re-infused with CAR-T or a normal person who has not been treated with CAR-T.

In an embodiment of the disclosure, the human proteins corresponding to the targets of the CAR are proteins such as CD19, BCMA, CD20, CD22, or CS1.

In an embodiment of the disclosure, a number of 10⁵ or more CAR-expressing CHO cells required for each of the standard, the blank control and the sample to be tested is collected.

### BRIEF DESCRIPTION OF THE DRAWINGS

Illustrative embodiments of the present disclosure are described in detail below with reference to the attached drawing figures.

FIG. 1 is a standard curve established by the standard of known protein molar concentration according to an embodiment of the disclosure.

FIG. 2 is a drawing showing a comparison of CAR copy number and anti-CAR antibody detection value in patient 1 according to an embodiment of the disclosure.

FIG. 3 is a drawing showing a comparison of CAR copy number and anti-CAR antibody detection value in patient 2 according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

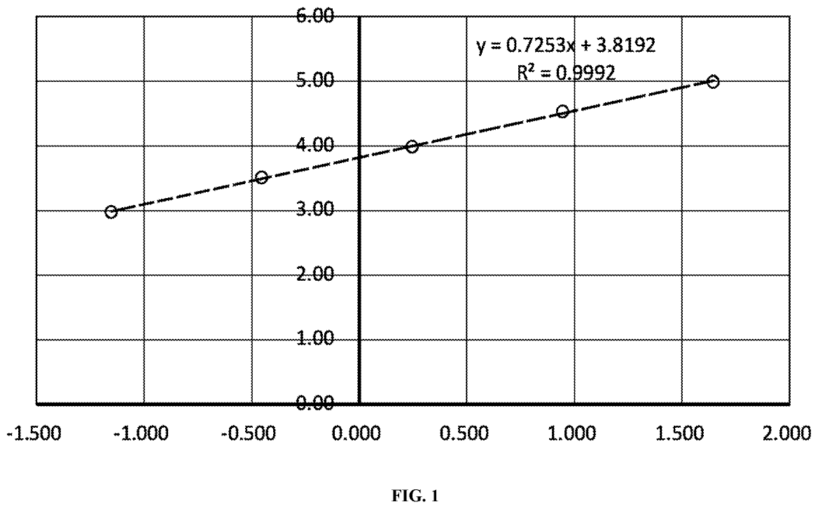

Referring to FIGS. 1-3, the disclosure provides a method for detecting an anti-CAR antibody against humanized CAR T cells, including detecting the anti-CAR antibody in a sample from a patient re-infused with humanized anti-CD19 CAR T cells from a patient and determining the detection limit.

Reagents and Materials

DMEM, phosphate buffered saline (PBS), trypsin cell digestion solution, and fetal bovine serum (FBS). Humanized anti-CD19 CAR lentivirus, human CD19 protein, PE-conjugated anti-human IgG Fc fragment antibody (IgG Fc-PE), and CHO cells.

Steps

Step S1, Culturing of target cell: humanized anti-CD19 CAR lentivirus was used to transfect CHO cells, and a large number of CHO cells expressing CAR (CAR-CHO) were obtained.

Step S2, Sample collection: 1 ml of venous blood was collected from normal people and patients using blood collection tubes containing EDTA anticoagulant, and the venous blood was centrifuged to obtain plasma, and the plasma was frozen at −70° C. until use. Repeated freezing and thawing should be avoided.

Step S3, Preparation of standard solution and blank control: human CD19 protein was prepared using PBS solution to produce a 100 ug/ml CD19 protein solution, and then the CD19 protein solution was gradiently diluted to create standard solutions No. 1 to 5 having a concentration of 2.5, 0.5, 0.1, 0.02, 0.004 ug/ml, respectively. The blank control was incubated without adding CD19 protein.

Step S4, Calculation of the number of CAR-CHO cells required for the standard solution, blank control, and sample: the tested articles were taken out and washed once with PBS solution and centrifuged. The supernatant was removed, then an appropriate amount of PBS solution was added to re-suspend the cells, resulting solution was aliquoted into 1.5 ml EP tubes for corresponding number of samples.

Step S5, Capture of anti-CAR antibody: 20 μl of the standard solution, the blank control and the plasma sample were added to corresponding EP tubes, mixed with CAR-CHO cells, and incubated at 4° C. for 30 minutes.

Step S6, PBS solution was added to the EP tubes to wash the cells once and centrifuged. The supernatant was collected and an appropriate amount of fresh PBS solution and 5 μl IgG Fc-PE antibody (0.5 mg/ml) was added to each tube. The resulting solutions were mixed well and incubated at 4° C. for 15 minutes.

Step S7, 1 ml of PBS solution was added to each tube to wash once and centrifuged, and the supernatant was removed. The residue was re-suspended in PBS solution, and the PE channel of the flow cytometer was used to detect the average fluorescence intensity of CAR-CHO cells in each tube.

Step S8, Calculation of concentrations of the standard curve and the anti-CAR antibody sample:

Step S8-1, Establishment of standard curve for CD19 protein.

The protein molar concentration (nM) of each standard was calculated by using the molecular weight of human CD19 protein and the protein concentrations of standards 1-5.

The average fluorescence intensity of the blank was subtracted from the average fluorescence intensity of all standards to obtain the corresponding ΔMFI, and the protein molar concentration and ΔMFI of standards 1 to 5 were converted into 1 g values. The 1 g values then were fitted into a linear standard curve to obtain the formula: y=0.7253x+3.8192. In this formula, x is the 1 g value of protein molar concentration, and y is the 1 g value of ΔMFI.

TABLE 1

| protein molar | CD19 | | | |
|---|---|---|---|---|
| concentration | concentration | | 1 g molar | |
| (nM) | (ug/ml) | ΔMFI | concentration | 1 g MFI |
| 0.071 | 0.004 | 933 | −1.151 | 2.97 |
| 0.353 | 0.02 | 3252 | −0.452 | 3.51 |
| 1.767 | 0.1 | 9600 | 0.247 | 3.98 |
| 8.834 | 0.5 | 34231 | 0.946 | 4.53 |
| 44.170 | 2.5 | 98529 | 1.645 | 4.99 |

Table 1: Determination and calculation results for standards 1 to 5, which was used to determine the standard curve.

As shown in FIG. 1, in this standard curve, R2=0.9992.

Step S8-2, Calculation of concentration of anti-CAR antibody in the sample:

As mentioned above, the protein molar concentration and ΔMFI of all samples to be tested were converted into 1 g values, and the 1 g values were substituted into the standard curve formula, and finally the molar concentration of anti-CAR antibody of each sample was calculated.

Step S9, Calculation of the negative reference value of the method: 10 different normal human plasma samples were simultaneously measured. After calculation of the molar concentration of anti-CAR antibody in normal people, the normality of the distribution of anti-CAR antibody concentration in the sample was firstly evaluated. After determination of the normal distribution data, the one-sided 95% confidence interval (right side) of the concentration of anti-CAR antibody in 10 normal human samples was calculated, the calculation results was thus the negative reference value of the anti-CAR antibody concentration, and the sample was determined as the sample from a patient that had been received corresponding CAR T cells if this reference value was exceeded.

Table 2: Determination results for anti-CAR antibody in normal human samples, and the negative reference value was 0.469 nM by using the values as shown below.

TABLE 2

| | Anti-CAR antibody concentration |
|---|---|
| Sample from Normal person | (nM) |
| 1 | 0.303 |
| 2 | 0.252 |
| 3 | 0.515 |
| 4 | 0.336 |
| 5 | 0.159 |
| 6 | 0.300 |
| 7 | 0.393 |
| 8 | 0.284 |
| 9 | 0.332 |
| 10 | 0.221 |

To sum up, a conclusion may be drawn.

The detection of anti-CAR antibodies in patients, combined with the changes in the copy number of CAR in patients, may help us to understand whether anti-CAR antibodies really affect the survival of CAR T cells and predict the possibility of recurrence in advance.

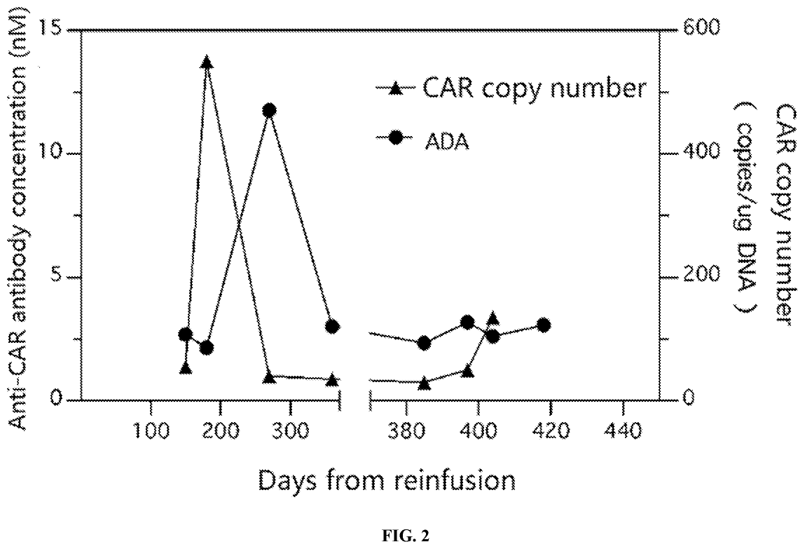

Patient 1 was found to have a recurrence during the 1-year follow-up, as shown in FIG. 2. It can be seen that the CAR copy number in patient 1 decreased significantly during the 9-month follow-up period compared with before, and the anti-CAR antibody at this time increased significantly, and it was believed that the neutralization effect of anti-CAR antibodies on CAR had an impact on the survival of CAR T cells, leading to a sudden decrease in CAR T cells and eventual relapse.

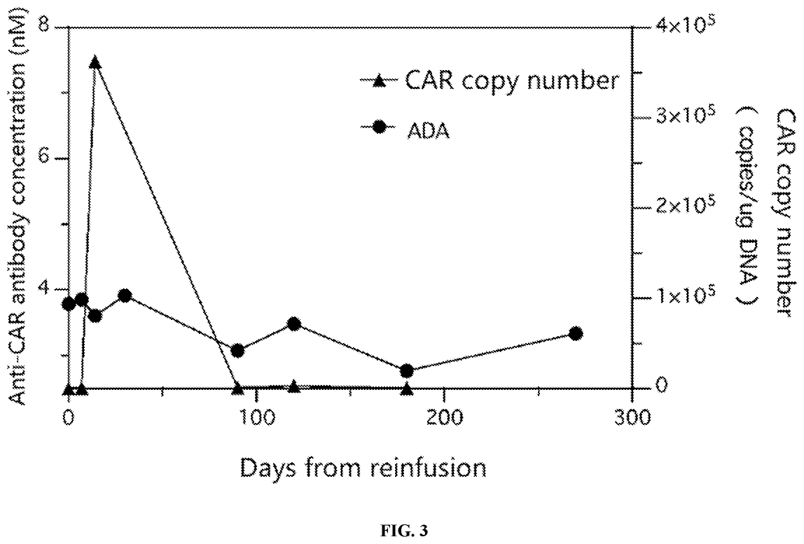

Patient 2 had not relapsed until the most recent follow-up, as shown in FIG. 3. It can be seen that the concentration of anti-CAR antibody in this patient was relatively stable, without significant changes, and the level was low. Therefore, the anti-CAR antibody was not the reason for affecting the survival of CAR T cells, nor did it cause recurrence.

The anti-CAR antibody detection values of the two patients were higher than the negative reference value, and the negative reference value was determined to be valid.

Quantification: in the proposal of the disclosure, the human protein corresponding to the target of the humanized CAR is used as a standard with a known molar concentration, so that it firstly binds to the CHO cells transfected with CAR, and then the PE-conjugated anti-human IgG Fc fragment antibody binds to the CAR-CHO-target protein complex, and a standard curve is established with the average fluorescence intensities measured by flow cytometry.

Human proteins are available on the market, and the corresponding molar concentration may be calculated from the mass concentration and the known molecular mass of the protein. The molar concentration is not limited by the molecular mass of the protein, and the concentration of anti-CAR antibodies may be quantified. In addition, the quantification may reach an nM level, while maintaining precision and specificity.

Simplicity and universality: the materials and technical methods such as cell transfection and flow cytometry used in this technical solution are routine laboratory materials and methods. The reagent and consumables are readily available and the method is easily operable. Small sample volume is required. The method of the disclosure is applicable to various humanized CARs and targets, as long as the lentiviral reagent for CAR transfection and the corresponding human target protein are replaced. The method may be applied mechanically. The method of the disclosure is generally applicable to laboratories or institutions that involve in the research and development of new CAR-T independently.

Although the embodiments of the present disclosure have been shown and described, those skilled in the art can understand that various changes, modifications and substitutions and variations can be made to these embodiments without departing from the principle and spirit of the present disclosure. The scope of the present disclosure is defined by the appended claims and their equivalents.

Various embodiments of the disclosure may have one or more of the following effects. In some embodiments, the disclosure may provide a method for detecting an anti-CAR antibody against humanized CAR T cells, which may allow for quantitative and regular monitoring the anti-CAR antibody, and timely reflecting the specific immune response of patients to CAR T cells. In other embodiments, the method of the disclosure may help researchers keep abreast of the efficacy of CAR T cells and make improvements, facilitate the understanding of the changes in the curative effect and continuous effect of CAR-T after re-infusion in patients in actual clinical work and the monitoring of recurrence possibility in a timely manner through the analysis of expression of anti-CAR antibodies, and be beneficial to make subsequent clinical decisions quickly and effectively.

Compared with the prior art, embodiments of the disclosure may have one or more of the following effects. In some embodiments, the technical proposal may allow for quantitative and regular monitoring of anti-CAR antibodies and timely reflection of the specific immune response of patients to CAR-T. In other embodiments, the disclosure may help researchers understand the efficacy of CAR T cells in a timely manner and make improvements. In clinical works, timely understanding of the changes in the curative effect and sustained effect of CAR-T after re-infusion, and timely monitoring of the possibility of recurrence through the analysis of expression of anti-CAR antibodies may be beneficial to making follow-up clinical decisions quickly and effectively. In further embodiments, the technical proposal of the disclosure may be cost-effective, simple, and fast to operate. It may allow for detection of a large number of samples at the same time and it may be suitable for promotion and application.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A method for detecting an anti-chimeric antigen receptor (CAR) antibody against humanized CAR T cells, comprising:
step S1, transfecting a humanized CAR into Chinese hamster ovary (CHO) cells using lentivirus to generate CHO cells expressing CAR as target cells;
step S2, collecting samples to be tested;
step S3, preparing different concentrations of standards using human proteins corresponding to a target of the CAR and establishing a standard curve;
step S4, incubating the target cells with the different concentrations of standards and the samples to be tested, and capturing an anti-CAR antibody;
step S5, incubating a phycoerythrin (PE)-conjugated anti-human IgG Fc antibody with the CHO cells for labeling; and
step S6, calculating a concentration of the anti-CAR antibody by detecting average fluorescence intensities of the standards, blank controls, and the samples to be tested using flow cytometry.

2. The method according to claim 1, wherein the step S2 comprises:
collecting 1 ml of venous blood from at least one group of normal people and patients using a blood collection tube containing EDTA anticoagulant;
conducting centrifugation of the venous blood to obtain plasma; and
freezing the plasma at −70° C. until use, without repeated freezing and thawing.

3. The method according to claim 1, wherein the step S4 comprises:
adding 20 ul of a standard, a blank control, and a plasma to corresponding EP tubes;
mixing with CAR-CHO cells; and
incubating a resulting mixture at 4° C. for 30 minutes.

4. The method according to claim 1, wherein the step S5 comprises:
adding PBS solution in an EP tube to wash once;
conducting centrifugation and discarding a resulting supernatant;
adding an amount of new PBS solution and 5 ul IgG Fc-PE antibody (0.5 mg/ml) to each tube; and
mixing well and conducting incubation at 4° C. for 15 minutes.

5. The method according to claim 1, wherein the step S6 comprises:
calculating a protein molar concentration (nM) of each standard using molecular weight of human CD19 protein and protein concentrations of standards 1 to 5;
subtracting an average fluorescence intensity of a blank sample from an average fluorescence intensity of all standards to obtain corresponding ΔMFI;
converting protein molar concentrations and ΔMFI of standards 1 to 5 into a first group of 1 g values;
fitting the first group of 1 g values into a linear standard curve to create a formula: y=0.7253x+3.8192, wherein x is a 1 g value of protein molar concentration and y is a 1 g value of ΔMFI;
converting all the protein molar concentration and ΔMFI of all samples to be tested into a second group of 1 g values and substituting the second group of 1 g values into the formula; and
calculating a molar concentration of anti-CAR antibody for each sample.

6. The method according to claim 1, wherein:
the humanized CAR is selected from various humanized CARs; and
targets comprises one or more proteins selected from the group consisting of CD19, BCMA, CD20, CD22, and CS1.

7. The method according to claim 1, wherein a sample to be tested comes from:
a subject who has been re-infused with CAR T cells; or
a normal person who has not been treated with CAR T cells.

8. The method according to claim 1, wherein the human proteins corresponding to the target of the CAR comprises one or more proteins selected from the group consisting of CD19, BCMA, CD20, CD22, and CS1.

9. The method according to claim 1, wherein a number of 10⁵ or more of CAR-expressing CHO cells required for each of a standard, a blank control, a the sample to be tested is collected.
